(19) United States
(12) Patent Application Publication
(10) Pub. No.: US 2013/0216594 A1
(43) Pub. Date: Aug. 22, 2013

(54) PREPARATION OF ORODISPERSIBLE FILMS

(71) Applicants: Andreas Krekeler, Holzkirchen (DE); Dimitri Neumann, Holzkirchen (DE)

(72) Inventors: Andreas Krekeler, Holzkirchen (DE); Dimitri Neumann, Holzkirchen (DE)

(73) Assignee: HEXAL AG, Holzkirchen (DE)

(21) Appl. No.: 13/881,970

(22) PCT Filed: Oct. 27, 2011
(86) PCT No.: PCT/EP2011/068825

§ 371 Date: Apr. 26, 2013

(30) Foreign Application Priority Data
Oct. 28, 2010 (DE) 10 2010 049 706.1

(51) Int. Cl. A61K 9/70

(52) U.S. Cl. CPC A61K 9/7007
USPC 424/400; 514/415

(57) ABSTRACT

The invention relates to a process for preparing a composition, more particularly a pharmaceutical composition for oral administration, comprising the steps of forming a suspension of at least one pharmaceutical ingredient and a solvent or solvent mixture, the at least one pharmaceutical ingredient being insoluble or poorly soluble in the solvent or solvent mixture, the step of adding at least one gel former to the suspension, the at least one gel former being swellable in the solvent or solvent mixture, and, optionally, the step of swelling the suspension.

### CROSS REFERENCE TO RELATED APPLICATIONS

This application is entitled to the benefit of and incorporates by reference essential subject matter described in International Patent Application PCT/EP2011/068825, filed Oct. 27, 2011, and German Patent Application 10 2010 049706.1, filed Oct. 28, 2010.

### Field of the Invention

The present invention relates to a process for preparing a pharmaceutical composition which is in particular suitable for the formation of an orodispersible film as well as a composition, in particular in the form of an orodispersible film.

### BACKGROUND OF THE INVENTION

The oral administration of drugs still represents the most widely used method of administration of drugs. For patient compliance with the oral administration human gustatory and olfactory sense are of great importance.

Traditional forms of administration are, for example, tablets or capsules, which are used as carriers for the oral administration of drugs. The tablets or capsules are in general swallowed, which requires that the patient holds a liquid, with which he can take this dosage form.

To some extent however, particularly in elderly persons and children there is discomfort in swallowing, such that those refuse the intake of tablets or capsules, or an intake only happens reluctant. This not infrequently results in poor compliance, which has adverse effects for the healing progress and the success of the therapy.

Even in groups of patients with mental illness, in which the monitoring of the actual intake of their medication is essential, the administration of conventional dosage forms such as tablets or capsules is not unproblematic. Due to delayed disintegration, such carriers of active ingredients can be easily removed from the mouth, without being notice by the supervising medical personnel.

To overcome the problems described, pharmaceutical dosage forms, such as for example granules or oral films have been developed, which can be taken without fluids and disintegrate rapidly in the oral cavity.

Oral films are characterized for example by the fact that they have a low thickness and a large surface and are able to disintegrate in short time in the oral cavity. Depending on the patient's needs, they can be taken anytime and anywhere and also discretely. A simultaneous intake of liquid is not necessary because the salivary fluid in the oral cavity is sufficient to dissolve the film and release the drug.

Oral films containing pharmaceutical and non-pharmaceutical active ingredients, and processes for their preparation are described, for example in WO 2007/009800, WO 2007/009801 and WO 03/011259.

A particular problem in the administration of conventional oral films, at least containing a pharmaceutical active ingredient is on the one hand the often unpleasant and sometimes perceived as an extremely bitter taste of the incorporated therein ingredients, on the other hand the fact that the films manufactured in conventional methods regarding the absolute drug content have only a limited capacity.

This is due to the fact that in conventionally produced oral films the drug, if it should be provided in a high dose in the pharmaceutical, should first be dissolved in a solvent system in which the drug is highly soluble. Oral films including for example a high-dose, well water-soluble active agent are, in accordance with the processes known from prior art, dissolved in an aqueous solution system. Just herein it shows to be disadvantageous that oral films containing a well water-soluble active agent dissolved in an aqueous solvent, cannot be dried to form homogeneous laminates. This disadvantage is particularly evident when the active agent is to be present in a high concentration and homogeneously distributed in the film. The active agent partially or entirely dissolves in the residual matrix due to heat impact during drying and/or is forming a concentrated salt solution that can no longer be dried. It can also come to the formation of islands of the active agent in the film, such that a homogeneous distribution of the ingredients in the film forming laminate cannot be guaranteed.

It was an objective of the present invention to provide a process for preparing a pharmaceutical composition, wherein the composition is particularly suitable for oral administration, that has high concentrations of active agents and wherein the active agent is homogeneously distributed in the composition. It was a further objective to provide a composition, in particular in the form of an orodispersible film.

### SUMMARY OF THE INVENTION

The present invention relates to a process for preparing a composition, in particular a pharmaceutical or cosmetic composition, preferably a pharmaceutical or cosmetic composition for oral administration, preferably in the form of an orodispersible film, comprising the steps of:
(i) forming a suspension of at least one pharmaceutical ingredient (A) and
a solvent or solvent mixture (B), wherein the at least one pharmaceutical ingredient (A) is in the solvent or solvent mixture (B) poorly soluble or insoluble,
(ii) adding at least one gel former (C) to the suspension, wherein the at least one gel former (C) being swellable in the solvent or solvent mixture (B) and optionally,
(iii) swelling of the suspension.

The process may further comprise the steps of:
(iv) forming a film of the suspension obtainable from step (ii) or step (iii) and
(v) drying of the film.

Additionally, the process may comprise adding at least one other substance selected from the group consisting of flavoring agent, sweetener and plasticizer, wherein said at least one other substance is in the solvent or solvent mixture (B) readily soluble.

The pharmaceutical ingredient (A) may be a pharmaceutically active ingredient or a mixture comprising at least one pharmaceutically active ingredient and the at least one pharmaceutical ingredient (A) may comprise between 10 wt % and 80 wt %, preferably between 50 wt % and 70 wt %, and the gel former (C) may comprise between 5 wt % and 25 wt %, preferably 7.5 wt % each based on the dry weight of the composition.

If a plasticizer, flavoring agent, and/or sweetener are added, the proportions of the plasticizer is between 2 wt % and 15 wt %, preferably 7.5 wt %
of the flavoring agent is between 0 wt % and 10 wt %, preferably 7% by weight, and/or of
the sweetener is between 0 wt % and 5 wt %, preferably 4 wt % each based on the dry weight of the composition.

In the process of the invention, the solvent mixture (B) may comprise at least two solvents, wherein the proportion of solvent in the mixture is between 50% (v/v) to 100% (v/v) respectively 0% (v/v) to 50% (v/v), and may be selected from C1-C5 alkanols, in particular ethanol or isopropanol, acetone or mixtures thereof.

Preferably, the pharmaceutical ingredient (A) is readily soluble in an aqueous solvent system or aqueous solvent systems, or mixtures thereof.

The solvent (B) is preferably a solvent mixture comprising acetone and ethanol, in particular a solvent mixture comprising acetone and ethanol with a proportion of acetone between 50% (v/v) and 95% (v/v) and a proportion of ethanol between 5% (v/v) and 50% (v/v), related to the solvent mixture.

The gel former (C) may be selected from the group consisting of hydroxypropyl cellulose, hydroxypropyl methyl cellulose, methyl cellulose, Klucel®, Klucel®E, Klucel®L, Klucel®J, Klucel ®G, Klucel®M, Klucel®H, Klucel®EF, Klucel®LF, Klucel®JF, Klucel®GF, Klucel®MF, Klucel®HF, Klucel®EXF, Klucel®LXF, Klucel®JXF, Klucel®GXF, Klucel®MXF, Klucel®HXF, ethyl cellulose or mixtures thereof.

The pharmaceutical ingredient (A) may be selected from the group consisting of sumatriptan, sildenafil, acetylcysteine, ambroxol, citalopram, clopidogrel, losartan, mitrazapine, valproic acid, verapamil, pharmaceutically acceptable salts thereof or mixtures thereof, and in particular selected from sumatriptan succinate and sildenafil citrate.

In a preferred embodiment of the method of the invention, the pharmaceutical ingredient (A) is sumatriptan succinate or Sildenafil citrate in a concentration of at 30-70 wt %, preferably 50-65 wt %, related to the dry weight of the composition.

The plasticizer may be selected from the group consisting of glycerol, propylene glycol, dibutyl sebacate, triacetin, triethyl citrate and isopropyl myristate, and mixtures thereof, preferably selected from glycerol and propylene glycol.

The formation of the film may be done in a casting-, drawing-, extrusion-, or spraying procedure and the drying of the film by application of heat.

The film may have a wet film thickness of 400 μm to 600 μm, preferably of 460 μm to 490 μm and/or a dry film thickness of 100 μm to 200 μm, preferably from 140 μm to 180 μm.

The invention comprises a process for preparing a pharmaceutical composition, in particular a composition for oral administration, preferably in the form of an orodispersible film, comprising the steps of:
(i) providing a solvent or a solvent mixture (B),
(ii) adding at least one sweetener, at least one plasticizer, and optionally at least
one flavoring agent to the solvent or solvent mixture (B),
(iii) suspending at least one pharmaceutical ingredient (A) in the mixture from step (ii),
(iv) adding at least one gel former (C) swellable in the solvent or solvent mixture (B)
to the mixture from step (iii),
(v) allowing the suspension from step (iv) to swell,
(vi) forming a film from the suspension from step (v),
(vii) drying the film,
wherein the at least one pharmaceutical ingredient (A) is poorly soluble or insoluble in the solvent or solvent mixture (B), and wherein the solvent or solvent mixture (B) is preferably a mixture of C1-C5 alkanol and acetone.

The invention further comprises a pharmaceutical composition, in particular pharmaceutical composition in the form of an orodispersible film prepared by a method described above.

The pharmaceutical composition may have a residual content of the solvent respectively the solvents (B) of less than 5000 ppm, preferably of less than 2000 ppm, related to the dry weight of the film.

Additionally, the inventive pharmaceutical composition may be in the form of an oral film which is anhydrous or substantially anhydrous, the at least one pharmaceutical ingredient (A) may be readily soluble in water or in an aqueous mixture of solvents, including saliva, and is poorly soluble or insoluble in the solvent or solvent mixture (B) used for the preparation of the film, selected from C1-C5 alkanols, in particular ethanol or isopropanol, acetone or mixtures thereof.

The pharmaceutical composition may comprise at least one pharmaceutically active ingredient selected from the group consisting of sumatriptan, sildenafil, acetylcysteine, ambroxol, citalopram, clopidogrel, losartan, mitrazapine, valproic acid, verapamil, pharmaceutically acceptable salts thereof or mixtures thereof, and in particular selected from sumatriptan succinate and Sildenafil citrate,

the water content of the pharmaceutical composition may be less than 0.5% by weight, the residual solvent may be 2000 ppm or less and the proportion of the at least one pharmaceutically active ingredient may be between 50% and 70% by weight.

### DETAILED DESCRIPTION OF THE INVENTION

The present invention relates to a process for preparing a composition, in particular a composition for oral administration, wherein the composition comprises a pharmaceutical ingredient respectively a pharmaceutical active ingredient.

The inventive method comprises the steps of:
(i) forming a suspension of at least one ingredient (A) and a solvent or solvent mixture (B), wherein the at least one ingredient (A) is in the solvent or solvent
mixture (B) poorly soluble or insoluble,
(ii) adding at least one gel former (C) to the suspension obtainable from step (i)
wherein the at least one gel former is chosen in that way that it is swellable in the solvent or solvent mixture (B).

In a preferred embodiment the method further comprises a step of
(iii) swelling of the resulting suspension from step (ii)

Herein, the duration of swelling is determined by the gel former used and the intended use or purpose of the suspension or of the formed gel, respectively. Depending on the kind of gel former as well as the duration of swelling and the treatment of the suspension during the swelling process, for example by stirring or shaking, the properties of the formed gel or a film or the like formed therefrom, can be influenced. The composition may already be administered in the form of administration of a suspension, as a thickened, preferably gel-like suspension, as a gel or as a suspension- or gel-filled capsule, for example in the form of a hard or soft gelatin capsule.

In a preferred embodiment of the process a film is formed from the suspension.

The process then further comprises the steps of:
(iv) forming a film of the suspension obtainable from step (ii) or step (iii) and
(v) drying the film.

After this the thus formed film can be brought in the final film-respectively film segment form, for example in a round, any rounded, oval, elliptical, triangular, rectangular or square or rectangular or polygonal shape.

The pharmaceutical ingredient used in the inventive process and to be formulated is preferably a pharmaceutically active ingredient (herein also referred to as the active ingredient) or a mixture comprising at least one pharmaceutically active ingredient. Of course here it is also given the possibility that two or more pharmaceutical ingredients and/or pharmaceutically active ingredients may be present in the mixture, eventually in different proportions. It may, beside the at least one pharmaceutical or pharmaceutically active ingredient, however, be provided at least one further pharmaceutically non-active ingredient or other ingredient.

In the context of the present invention a pharmaceutical ingredient to be formulated denotes any materials and substances to be used in a pharmaceutical and/or a pharmaceutical-associated use, for example a cosmetic use or for dietetic purposes and which may be contained in respective compositions. These include for example also preservatives, dyes, vitamins, trace elements, nutrients, micro nutrients and humectants.

As a pharmaceutically effective ingredient in the context of the present invention, substances are designated that cause at a low dose a specific effect or a reaction in an organism. As pharmaceutically effective ingredient in the context of the present invention, substances are to be understood, to which a therapeutic effect is attributed. Apart from naturally occurring substances beneath these also semisynthetic derivatives and totally synthetic or biotechnologically produced chemical agents are understood. Besides, also adjuvants or active amplifier are incurred in connection with the present invention by this definition.

Before step (iv) formation of the film respectively after process step (ii) the addition of the gel former, optionally a (iii) swelling of the suspension can be processed. The suspension is allowed to swell in this case preferably for at least 12 hours, such that the gel former added to the suspension advantageously can swell completely. The duration of swelling is oriented according to the gel former used in each case and the desired degree of swelling. The latter depends on the intended use of the composition prepared in the inventive process. If the use is to be provided in the form of a gel, paste or the like, a shorter duration of swelling or a lower content of the gel former can be chosen to define the consistency of the composition. The choice thus influences the length of the swelling process. The swelling can take place under stirring or in stationary suspension. This also results in advantages for the film formation, this means for the formation of a homogeneous, orodispersible film having uniform structure and texture as well as uniform distribution of the ingredients, active pharmaceutical ingredients and/or pharmaceutically active ingredients or mixtures thereof.

The pharmaceutical or pharmaceutically active ingredient, respectively, is in an embodiment characterized in that it is readily soluble in an aqueous solution system or in aqueous solution systems or mixtures thereof. For example, the ingredient is readily soluble in water. Besides, the liquid present in the mouth of a patient is comprised by the mentioned aqueous solvents or solvent mixtures as well.

If the ingredient to be formulated is a highly water soluble ingredient, in the present invention solvent or solvent mixtures (B) are preferably used as solvent or solvent mixtures (B) which are selected from C1- to C5-alkanols and herein in particular, organic solvents, for example ethanol or isopropanol or acetone. Of course, mixtures of the aforementioned C1- to C5-alkanols, in particular ethanol or isopropanol and/or mixtures with acetone or pure acetone can be used.

In a further embodiment of the invention the ingredient to be formulated is insoluble or poorly soluble in an aqueous solvent (system). In this case the solvent or solvent mixture (B), respectively, is an aqueous solvent or solvent mixture, respectively, preferably water.

In each case, a solvent respectively solvent mixture is used, in which the ingredient to be formulated or the active ingredient, respectively, is insoluble or poorly soluble. It can thus be achieved that during the process of preparing the composition and in this case in particular in the formation of a orodispersible film the ingredient/active ingredient, does not dissolve in the solvent and is instead available in a dispersed form.

This prevents that the ingredient to be formulated in a possible drying of the composition forms a concentrated salt solution that cannot be dried any more. Moreover it is prevented that during drying of the composition non-uniform islands of active ingredient form in the composition, such that no homogeneous distribution of the active substance within the composition, for example over the entire orodispersible film is given.

The inventive method may further includes the addition of at least one further substance. This further substance is preferably selected from the group consisting of flavoring agents, sweetening agents, and plasticizers, wherein the at least one further substance is preferably readily soluble in the solvent or solvent mixture (B).

The terms “readily soluble” and “poorly soluble” or “insoluble” as used herein, are preferably derived from a classification of solubility, wherein the range of readily soluble substances is limited to a solubility of 0.1 to 1 mol/l and a solubility of <0.1 mol/l is considered as poorly soluble or insoluble. In this context it is considered favorable if the at least one pharmaceutical ingredient, the use of which is provided for in the process of this invention, has a solubility of <0.1 mol/l in the solvent or solvent mixture (B).

It is considered especially beneficial if the further substance(s) is/are added before, after or simultaneously with the pharmaceutical and pharmaceutically active ingredient to the solvent or solvent mixture (B). Flavoring, sweetener and/or plasticizers can be dissolved or dispersed, for example prior to the addition of the ingredient or gel former, in the solvent or solvent mixture (B) or in a mixture partner (B) of the solvent mixture (B) and/or dispersed. The mixture thus obtained can then, if appropriate after addition of one further or more further mixture partner(s) of the solvent mixture (B), be used as solvent for the suspension. Of course, further substances may be added to the suspension later, i.e. for example, after the addition of pharmaceutical ingredient and/or gel former.

In an advantageous development of the inventive method it is provided that the resulting composition is formed as an orodispersible film and therein the at least one ingredient is present in a weight fraction of 10% to 80% by weight, preferably from 50% to 70% by weight and particularly preferably of 65% by weight, related to the dry weight of the composition. Such high drug concentrations are not available with prior art methods, since this can result in the aforementioned problems when drying the films formed from the composition. The high-dosed drugs herein can form concentrated salt solutions, which can no longer be dried or it may result in an uneven distribution of the active ingredient.

With the process of this invention, the aforementioned high drug concentrations in the compositions, can be realized in particular in compositions for oral administration, preferably in the form of an orodispersible film without causing any adverse effects, such as the formation of a concentrated, non-dryable solution. In a particularly preferred embodiment in the inventive process readily water-soluble pharmaceutical ingredients are formulated, wherein preferably an organic solvent or solvent mixture (B) is used, which is free or substantially free of water and wherein said at least one pharmaceutical ingredient is in this solvent or solvent mixture (B) insoluble or poorly soluble, and spreads (disperses) uniformly throughout the suspension. The homogeneous distribution is maintained even during the drying of the composition in the form of a film. “Substantially anhydrous” in this context means a proportion of water in the solvent or solvent mixture (B) of less than 2% (v/v), preferably less than 0.5% (v/v).

In the process of the invention it is further provided that the proportion of the gel former is between 5% and 25% by weight, preferably between 10% and 15% by weight and more preferably at 7.5% by weight, wherein this value relates to the dry weight of the composition. With the proportion of gel former also film properties such as texture, flexibility and thickness can be defined.

The method of the invention further provides in a preferred embodiment that a plasticizer is employed, the proportion of which in the composition resulting from the method is between 2% and 15% by weight, preferably 7.5% by weight. The flavoring agent which also additionally or alternatively be used in the method preferably has a proportion of between 0% and 10% by weight, preferably of 7% by weight related to the dry weight of the composition.

If additionally or alternatively, a sweetener is used, its proportion is desirably between 0% and 5% by weight, preferably at 4% by weight related to the dry weight of the composition. The aforementioned flavoring agents or sweeteners are used to mask the taste or to increase the compliance of corresponding compositions, for example, in the form of orodispersible films and all other appropriate forms of administration, such as gels or (gelatin) capsules with gel filling, as they give these a particularly pleasant taste and/or mask an unpleasant taste.

The gel former used in the method is a gel former which is swellable in the solvent or solvent mixture (B), i.e. preferably in a solvent or solvent mixture selected from C1- to C5-alkanols, in particular of ethanol or isopropanol, acetone or mixtures thereof.

Particularly preferably, the gel former is selected from the group consisting of hydroxypropyl cellulose (HPC), hydroxypropyl methyl cellulose (HPMC) and methyl cellulose (MC) and mixtures thereof. Usable as gel formers are also the pharmaceutically acceptable gel formers for example known under the trade name Klucel, Klucel® E, Klucel® L, Klucel® J, Klucel® G, Klucel® M, Klucel® H or their respective freenesses designated by the names Klucel® EF, Klucel® LF, Klucel® JF, Klucel® GF, Klucel® MF, Klucel® HF, Klucel® EXF, Klucel® LXF, Klucel® JXF, Klucel® GXF, Klucel® MXF, Klucel® HXF as well as mixtures thereof. The group denoted by the trade name Klucel® hydroxypropyl cellulose is a non-ionic, water-soluble cellulose ether. Klucel® is soluble both in aqueous and in organic solvents and has thermoplasticity, surface activity, as well as thickening an stabilization properties similar to conventional water-soluble cellulose polymers. The invention is not limited to the products of Klucel® serie includes as well the use of comparable substances from other manufacturers.

Depending on the field of application of the composition produced by the method of the invention the suitable gel former is selected. Therewith texture as well as other properties of the gel in terms of flexibility, drying and suitability for film formation can be set or defined.

The ingredient which is poorly soluble or insoluble in the solvent or solvent mixture (B) used in the method of the invention, is preferably a pharmaceutically active ingredient, or a mixture comprising at least one pharmaceutically active ingredient as defined above.

A pharmaceutically active ingredient which can be formulated by the method of the invention is for example selected from the group consisting of sumatriptan, sildenafil, acetyl cysteine, ambroxol, citalopram, clopidogrel, losartan, mitrazapine, valproic acid, verapamil and/or pharmaceutically acceptable salts thereof.

Furthermore mixtures of the aforementioned ingredients and other substances are suitable. Said pharmaceutically active ingredients can be used in their pure form or as pharmaceutically acceptable salts. Besides, there is also the possibility to use the pure form of one or more of the aforementioned ingredients with a pharmaceutically acceptable salt of a further ingredient in the inventive method.

In an embodiment of the method considered as particularly advantageous as ingredient or pharmaceutically active ingredient sumatriptan or sildenafil citrate is used.

In this case preferably, the content of sumatriptan succinate and sildenafil citrate is between 30 wt % to 70 wt %, advantageously at 50% by weight to 65 wt % of active ingredient related to the dry weight of the composition. More preferably, the composition comprising sumatriptan succinate or sildenafil citrate composition is in the form of an orodispersible film. The process offers a particular advantage, as in conventional processes high concentrations of the aforementioned substances may not or hardly be achieved.

In the methods known to date for the formation of corresponding compositions in particular in the form of orodispersible films, that suggest the use of water as a solvent for an water-soluble active substance to be formulated, the maximum attainable proportion of active compound in the composition or in the film, especially of the aforesaid drugs sumatriptan succinate or sildenafil citrate is at most 10 wt %.

At higher concentrations the above-described negative effects especially in drying of the films occur, such that up to now no satisfactory film could be produced. Using corresponding percentages of sumatriptan succinate, for example in the range of 65 wt % and a solution of the active ingredient in an aqueous solvent it appears that with the known methods no films can be made. In contrast thereto in the anhydrous method of the invention, compositions can be provided which can be designed as oral films which can have proportions of active ingredient of 65 wt % and more, preferably of up to 80 wt %, without showing disadvantages known from prior art.

The plasticizer optionally used in the process of the invention serves to reduce the brittleness of the composition provided in the form of a film and to increase its flexibility. In a preferred embodiment of the inventive method, the plasticizers may be selected from glycerol and propylene glycol. Also suitable as plasticizers are dibutyl sebacate, triacetin, triethyl citrate and isopropyl myristate. The use of propylene glycol and glycerol is preferred.

The sweetener usable in the method is preferably selected from the group consisting of monosaccharides, disaccharides, polysaccharides, in particular maltodextrin, sucralose, neotame, alitame, cyclamate, sorbitol, xylitol, saccharin, aspartame, or mixtures of the aforesaid. By the amount or the percentage of sweetener, a flavoring of the composition or the orodispersible film can be made. The taste may, depending on the sweetener, be made more or less sweet, such that thereby also, due to its pleasant sweet taste, which is achieved by the addition of appropriate sweeteners, patient compliance can be increased.

Beside the addition of sweeteners, a preferred embodiment of the method provides that a flavoring agent in the above percentages is used in the method. The flavoring agent is preferably selected from the group consisting of flavorings with minty and fresh taste and here in particular peppermint oil, peppermint flavor, menthol, and/or levomenthol.

In addition, alternatively or additionally flavors with sour fresh taste, especially grapefruit flavor, lemon or orange flavor can be used in the inventive method. The taste of the composition, provided for example in the form of an orodispersible film and the taste sensation occurring after ingestion can thus be made comfortable for the patient. Beside the aforementioned flavoring agents or combinations of these, the flavoring agent can also be selected from flavoring agents with sweet, nutty taste, in particular from those flavoring agents that produce a nut flavor, a chocolate flavor, a cinnamon or spice flavor or the like. Naturally, there is also the possibility to use a mixture of the aforesaid with the flavoring agents as previously described. Besides the mentioned flavoring agents, hereby certainly any other suitable or consider expedient flavoring agents can be used to determine or define the flavor of respective compositions to thereby increase patient's compliance.

In the inventive method it is provided that the ingredient (A) is poorly soluble or insoluble in a solvent or solvent mixture (B). Is considered to be beneficial in this context, if the used solvent mixture (B) is a mixture of at least two solvents. The solvents in this case have desirably proportions of from 50% (v/v) to 100% (v/v), respectively from 0% (v/v) to 50% (v/v), each related to the solvent mixture (B). Depending on the solvent used, the proportions are determined, accordingly.

A preferred embodiment of the inventive method provides that the solvent or solvent mixture (B) consists of ethanol and isopropanol or mixtures thereof. The amount of ethanol or isopropanol in the solvent mixture (B) is between 0% (v/v) and 30% (v/v), and between 70% (v/v) and 100% (v/v), respectively. The corresponding mixture components and solvents used in the solvent mixture (B) depend on the content of the substance(s) (A) to be formulated. Besides the aforementioned alkanols ethanol and isopropanol, the solvent or solvent mixture (B) may also be acetone or comprise a proportion of acetone, respectively. A preferred embodiment provides that, as a solvent or solvent mixture (B) a mixture of acetone and ethanol is used. The proportion of acetone in the solvent mixture (B) is in this case in particular between 50% (v/v) and 95% (v/v), while the proportion of ethanol in the solvent mixture (B) is between 5% (v/v) and 50% (v/v). Naturally there is also the possibility that ethanol or acetone (v/v) with a proportion of 100% is used, respectively, and that a combination or a mixture of acetone and isopropanol is used as a solvent or solvent mixture (B). Herein the respective proportions of isopropanol and acetone are in the above range, namely between 50% (v/v) and 95% (v/v) acetone and a proportion of 5% (v/v) to 50% (v/v) isopropanol, each related to the solvent mixture (B).

The inventive method also provides a step of forming a film. The formation of the film is preferably carried out in a casting, extrusion or spray method. The suspension obtainable from step (ii) or (iii) of the inventive method, comprising at least one ingredient (A) and at least one gel former is subjected to a film formation process considered favorable. The suspension is, for example, cast on a corresponding support surface on which the suspension distributes homogeneously. By the cast amount and the casting surface the wet film thickness of the film is set, before the latter is then subjected to a drying process, for example by heating the casting surface. The same applies to the use of drawing or extrusion method. If a spray method is used, the suspension is sprayed onto a respective carrier. By the sprayed amount and the duration of spraying hereby the wet layer thickness of the film is specifically set before it is then subjected to a drying process.

Preferably, the drying of the film is done by application of heat. The application of heat can for example be done by a hot air blower. There is of course also the possibility that the film is dried in an oven or a comparable drying device.

In the inventive method advantageously a film with a wet film thickness of 400 to 600 μm, preferably of 460 to 490 μm can be produced. The film can then preferably have a dry film thickness of 100 to 200 μm, preferably of 140 to 180 μm. Of course, the thickness of the film can also be defined through the dry film thickness, in which case the wet layer thickness is extraneous. The dry film thicknesses preferably provided are between 100 and 200 μm, preferably of between 140 and 180 μm. A dry film thickness within the mentioned limits ensures rapid disintegration of the film within the oral cavity. This ensures that the drug is completely absorbed by the patient. Of course also laminates with lower or higher layer thicknesses can be provided, which is encompassed by the invention as well.

The inventive method has the following advantages. The ingredient shows, due to the solvent used, no or only a very low solubility in the suspension. If the invention method provides a drying step, while drying the composition, the formation of islands of active ingredient or non-dryable accumulations of substance, which prevent the homogenous distribution of the active ingredient in the composition, are avoided. When using anhydrous solvents or solvent mixtures, respectively, and hereby preferably acetone or acetone-ethanol, acetone-isopropanol or ethanol-isopropanol-acetone mixtures, low drying temperatures compared to water-based compositions, particularly in the form of orodispersible films, can be achieved. This has the advantage that lower thermal loads on the component(s) in particular the pharmaceutically active ingredients occur and no loss of active ingredient is to be reported. The low drying temperatures have a positive effect on the essential oils and other flavorings used in the method, as those by reducing the thermal exposure tend to less decay and reduction.

Due to the very homogeneous distribution of pharmaceutical or pharmaceutically active ingredient in the composition that can be prepared by the method of the invention, in the case of the production of orodispersible films high coating or film formation rates are possible since drug agglomeration in the suspensions, arising for example in water-based solvents and substances dissolved therein, do not need to be considered. In the inventive method also significantly thinner films compared to aqueous systems can be achieved, thus patient compliance increases, as correspondingly thinner films can be more easily be taken up and disintegrate faster. Another advantage of the inventive method is the fact that the active ingredient in orodispersible film is provided in original, non-dissolved form. It follows therefrom that the bitter taste occuring in water-soluble drugs or their solution and subsequent drying in aqueous systems, respectively, is pushed back. The pharmaceutically active ingredient is not provided amorphously, such that the bitter taste impression is alleviated. This offers the advantage that excessive use of taste masking agents can be avoided, which in turn significantly increases patient compliance.

An embodiment of the inventive method considered especially favorable comprises the steps of:
Dissolving at least one flavoring agent in a first mixture component of a solvent mixture (B), for example in ethanol, isopropanol or acetone,
Adding a second mixture component of the solvent mixture (B) to the mixture of at least one flavoring agent, and the first mixture component of a solvent mixture (B),
Dissolving a sweetener and a plasticizer in the mixture of first mixture component, the second mixture component and flavoring agent,
Suspending of a pharmaceutical or a pharmaceutically active ingredient in the mixture of the first mixture component of the solvent mixture, the second mixture component of the solvent mixture, flavoring agent, sweetener and plasticizers,
Adding a gel former swellable in the solvent mixture (B) to the mixture of the first mixture component of the solvent mixture (B), the second mixture component of the solvent mixture (B), flavoring agent, sweetener, plasticizer and pharmaceutical or pharmaceutically active ingredient,
Swelling of the suspension,
forming a film and
drying of the film.

More preferably, the solvent or solvent mixture (B) is anhydrous or substantially anhydrous, wherein “substantially anhydrous” means a water content of the solvent or solvent mixture (B) of less than 2% (v/v), preferably of less than 0.5% (v/v).

The invention also relates to a composition, particularly a composition in the form of an orodispersible film. The composition comprises at least one pharmaceutical ingredient as defined above and at least one gel former as defined above. The composition is characterized in that it is readily soluble preferably in aqueous solvent systems, including saliva, and thus quickly disintegrates when used in the oral cavity.

The pharmaceutical ingredient used in the composition is preferably at least one pharmaceutically active ingredient as defined above, or mixture as defined above comprising at least one pharmaceutically active ingredient. The pharmaceutical ingredient or pharmaceutically active ingredient in this case preferably has in the solvent or solvent mixture (B) used in the preparation of the composition a solubility of less than 0.1 mol/l (poorly soluble or insoluble).

The composition of the invention preferably comprises a water-soluble pharmaceutically active ingredient. Also preferably the composition of the invention is free or substantially free of water and of the solvent or the solvent mixture (B) used for the preparation. The proportion of solvent or solvent mixture (B) in the final composition is less than 20000 ppm, preferably less than 10000 ppm and most preferably less than 5000 ppm. In particular, the proportion of solvent or solvent mixture (B) is at 2000 ppm or less.

Preferably, the composition of the invention is a solid composition and in particular in this case a solid composition in the form of a single-layer film. Further preferably, such a composition rapidly disintegrates after contact with an aqueous solution, such as saliva. Thereby single-layer means that the film is in the form of a single layer, wherein the layer is preferably homogeneous. The film thereby may be flexible or non-flexible. Preferably, the film, also due to the added plasticizers, is flexible and therefore adapts well to the shape of the oral cavity.

The composition of the invention in the form of an orodispersible film may be present as round, arbitrarily rounded, oval, elliptical, triangular, quadrangular or rectangular, respectively or square or polygonal film. The disintegration time of the inventive composition in the oral cavity is 1 to 100 seconds, preferably 1 to 50 sec, preferably 1 to 10 sec.

### EXAMPLES

Production Method

### Example 1

| | | |
|---|---|---|
| Sumatriptan-succinate | 65 | g |
| Grapefruit flavor | 6.8 | g |
| Levomenthol | 0.2 | g |
| Sucralose | 4 | g |
| Glycerol | 4 | g |
| Klucel ® LF | 20 | g |
| | 100 | g |
| Solvents | | |
| Ethanol*⁾ | 93.6 | g |
| Acetone*⁾ | 40.1 | g |

*⁾is almost completely removed during production

Preparation of the Coating Mass:

Grapefruit flavor and levomenthol are initially dissolved in acetone, then ethanol is added and sucralose and propylene glycol also dissolved in the mixture. Next, the active ingredient is mixed into the suspension and finally the gel former Klucel® LF is added. The mass is allowed to swell for at least 12 hours. After the swelling process, a suitable coating device is coated with the swollen suspension at 0.7 m/min and the composition is dried to form a laminate at a drying temperature of maximum 40° C.

In the illustrative embodiment, a mixture of two solvents is used. These are the organic solvents acetone and ethanol. The proportion of ethanol in this case is 93.6 g, while 40.1 g acetone as solvent or mixture component is used. After drying, the residual solvent levels in the orodispersible films each are 2000 ppm for ethanol and acetone.

Regulatory permitted are residual solvent contents of 5000 ppm.

### Example 2

| | | |
|---|---|---|
| Sumatriptan-succinate | 65 | g |
| Grapefruit flavor | 6.8 | g |
| Levomenthol | 0.2 | g |
| Sucralose | 4 | g |
| Glycerol | 5 | g |
| Klucel ® LF | 19 | g |
| | 100 | g |
| Solvents | | |
| Ethanol*⁾ | 98.8 | g |
| Acetone*⁾ | 42.3 | g |

*⁾is almost completely removed during production

Preparation of the Coating Mass:

Grapefruit flavor and levomenthol are initially dissolved in acetone, then ethanol is added and sucralose and propylene glycol also dissolved in the mixture. Next, the active ingredient is mixed into the suspension and finally the gel former Klucel® LF is added. The mass is allowed to swell for at least 12 hours. After the swelling process, a suitable coating device is coated with the swollen suspension at 0.7 m/min and the composition is dried to form a laminate at a drying temperature of maximum 40° C.

In the illustrative embodiment, a mixture of two solvents is used. These are the organic solvents acetone and ethanol. The proportion of ethanol in this case is 98.8 g, while 42.3 g acetone as solvent or mixture component is used. After drying, the residual solvent levels in the orodispersible films each are 2000 ppm for ethanol and acetone.

Regulatory permitted are residual solvent contents of 5000 ppm.

### Example 3 (Not Executable Comparative Example)

| | | |
|---|---|---|
| Sumatriptan-succinate | 65 | g |
| Grapefruit flavor | 6.8 | g |
| Levomenthol | 0.2 | g |
| Sucralose | 4 | g |
| Glycerol | 7.5 | g |
| HPMC (metholose SM-100) | 9 | g |
| NaCl | 2 | g |
| Pullulan | 5 | g |
| Tween 80 | 0.5 | g |

Preparation of the Coating Mass:

In this illustrative embodiment mentioned as comparative example it is shown that similar proportions of pharmaceutically active ingredient can only be achieved by the method described in the invention. In the illustrative embodiment mentioned as example 3 pullulan is dissolved in the aqueous Tween-solution. Levomenthol and grapefruit flavor are dissolved separately in a portion of ethanol. The residual amount of ethanol is added to the aqueous pullulan solution. Then the flavor solution is added. Subsequently the ingredient glycerol and sucralose are added. Lastly HPMC is stirred in. The mass is also, as previously described for Example 1 and 2, allowed to swell for 12 hours. Then it appeared that even at drying temperatures of up to 80° C. a peelable and further processable laminate, serving as a basis for an orodispersible film cannot be produced.

What is claimed is:

1. A method for preparing a pharmaceutical composition for oral administration comprising the steps of:
(i) forming a suspension of at least one pharmaceutical ingredient (A) and a solvent or a solvent mixture (B), wherein the at least one pharmaceutical ingredient (A) is poorly soluble or insoluble in the solvent or solvent mixture (B),
(ii) adding at least one gel former (C) to the suspension, wherein the at least one gel former (C) is swellable in the solvent or solvent mixture (B) and, optionally
(iii) allowing the suspension to swell.

2. The method according to claim 1, further comprising the steps of:
(iv) forming a film of the suspension obtainable from step (ii) or step (iii) and
(v) drying the film.

3. The method according to claim 1, further comprising adding of at least one further substance selected from the group consisting of flavoring agent, sweetener and plasticizer, wherein said at least one further substance is readily soluble in the solvent or solvent mixture (B).

4. The method according to claim 1, wherein
the pharmaceutical ingredient (A) is a pharmaceutically active ingredient or a mixture comprising at least one pharmaceutically active ingredient.

5. Method The method according to claim 1, wherein
the at least one pharmaceutical ingredient (A) is between 10% and 80% by weight, and
the gel former (C) is between 5% and 25% by weight,
each related to the dry weight of the composition.

6. The method according to claim 3, wherein
the plasticizer is between 2% and 15% by weight,
the flavoring agent is between 0% and 10% by weight, and
the sweetener is between 0% and 5% by weight,
each related to the dry weight of the composition.

7. The method according to claim 1, wherein
the solvent mixture (B) comprises at least two solvents, wherein the proportion of solvent in the mixture is between 50% (v/v) to 100% (v/v) respectively 0% (v/v) to 50% (v/v).

8. The method according to claim 7, wherein
the pharmaceutical ingredient (A) is readily soluble in an aqueous solvent system or aqueous solvent systems, or mixtures thereof, and the solvent or solvent mixture (B) is selected from C1-C5 alkanols, acetone or mixtures thereof.

9. The method according to claim 8, wherein
the pharmaceutical ingredient (A) is readily soluble in an aqueous solvent system or aqueous solvent systems, or mixtures thereof, and the solvent (B) is a mixture of solvents comprising acetone and ethanol.

10. The method according to claim 1, wherein
the gel former (C) is selected from the group consisting of hydroxypropyl cellulose, hydroxypropyl methyl cellulose, methyl cellulose, Klucel®, Klucel® E, Klucel® L, Klucel® J, Klucel® G, Klucel® M, Klucel® H, Klucel® EF, Klucel® LF, Klucel® JF, Klucel® GF, Klucel® MF, Klucel® HF, Klucel® EXF , Klucel® LXF, Klucel® JXF, Klucel® GXF, Klucel® MXF, Klucel® HXF, ethyl cellulose or mixtures thereof.

11. The method according to claim 1, wherein
the pharmaceutical ingredient (A) is selected from the group consisting of sumatriptan, sildenafil, acetylcysteine, ambroxol, citalopram, clopidogrel, losartan, mitrazapine, valproic acid, verapamil, and pharmaceutically acceptable salts thereof or mixtures thereof.

12. The method according to claim 11, wherein
the pharmaceutical ingredient (A) is selected from sumatriptan succinate or sildenafil citrate comprising 30-70% by weight, related to the dry weight of the composition.

13. The method according to claim 3, wherein
the plasticizer is selected from the group consisting of glycerol, propylene glycol, dibutyl sebacate, triacetin, triethyl citrate and isopropyl myristate as well as mixtures thereof, preferably selected from glycerol and propylene glycol.

14. The method according to claim 2, wherein
the formation of the film is done by casting-, drawing-, extrusion- or spraying procedure and the drying of the film is done by application of heat.

15. The method according to claim 2, wherein
the film has a wet film thickness of 400 μm to 600 μm, preferably of 460 μm to 490 μm and a dry film thickness of 100 μm to 200 682 m, preferably of 140 μm to 180 μm.

16. A method for preparing a pharmaceutical composition, for oral administration, in the form of an orodispersible film, comprising the steps of:
(i) providing a solvent or a solvent mixture (B),
(ii) adding at least one sweetener, at least one plasticizer, and optionally at least
one flavoring agent to the solvent or mixture of solvents (B),
(iii) suspending at least one pharmaceutical ingredient (A) in the mixture from step (ii),
(iv) adding at least one gel former (C) swellable in the solvent or solvent mixture (B) to the mixture from step (iii),
(v) allowing the suspension from step (iv) to swell,
(vi) forming a film from the swelled suspension from step (v), and
(vii) drying the film,
wherein the at least one pharmaceutical ingredient (A) is poorly soluble or insoluble in the solvent or solvent mixture (B), and wherein the solvent or solvent mixture (B) is a mixture of a C1-C5 alkanol and acetone.

17. A pharmaceutical composition prepared by the method according to claim 1.

18. The pharmaceutical composition according to claim 17, wherein the residual content of the solvents (B) is less than 5000 ppm related to the dry weight of the film.

19. The pharmaceutical composition according to claim 18, wherein
the pharmaceutical composition is in the form of an oral film which is substantially anhydrous and that at least one pharmaceutical ingredient (A) is readily soluble in water or in an aqueous mixture of solvents, including saliva, and the solvent mixture (B) used for the preparation of the film, selected from C1-C5 alkanols, acetone and mixtures thereof.

20. A pharmaceutical composition prepared from a solvent comprising a mixture of ethanol and acetone or a mixture of isopropanol and acetone and comprising at least one pharmaceutically active ingredient selected from the group consisting of sumatriptan, sildenafil, acetylcysteine, ambroxol, citalopram, clopidogrel, losartan, mitrazapine, valproic acid, verapamil, and pharmaceutically acceptable salts thereof or mixtures thereof, wherein the water content of the pharmaceutical composition is less than 0.5% by weight, the residual solvent content is 2000 ppm or less, and the proportion of the at least one pharmaceutically active ingredient is between 50% and 70% by weight.
